(19) United States
(12) Patent Application Publication
(10) Pub. No.: US 2010/0275955 A1
(43) Pub. Date: Nov. 4, 2010

(54) METHOD AND DEVICE FOR STERILIZING AND WASHING CAP

(76) Inventors: Nobuyuki Ueda, Yokohama (JP); Satoshi Uehara, Yokohama (JP); Kenichi Kominami, Yokohama (JP); Yuusuke Suda, Yokohama (JP)

Correspondence Address: Cheng Law Group, PLLC, 1100 17th Street, N.W., Suite 503, Washington, DC, 20036, US

(21) Appl. No.: 12/810,385

(22) PCT Filed: Dec. 2, 2008
(86) PCT No.: PCT/JP2008/071841

§ 371 Date: Jun. 24, 2010

(30) Foreign Application Priority Data
Dec. 28, 2007 (JP) 2007-338698

(51) Int. Cl. B08B 3/02; B65B 55/00; A61L 2/00

(52) U.S. Cl. 134/25.4; 134/131

(57) ABSTRACT

A chute type sterilizing and washing device for a cap, in which deformation of a cap is prevented by relaxing integral pressure acting on a synthetic resin cap in the chute, and sterilization and washing can be carried out efficiently by reducing the impact of hot water on germ-free water jetted at the upstream side on the hot water or germ-free water at the downstream side. The chute (5) has ascending chute portions (7, 9), a hot water pipe having a plurality of nozzles for jetting out hot water as a sterilizing and washing medium is arranged along the ascending chute portion, hot water is jetted to a cap fed to the ascending chute portion, the cap (35) receives a thrust for ascending the ascending shoot portion by the jet pressure of the hot water thus sterilizing and washing the cap with the sterilizing and washing medium and conveying.

### TECHNICAL FIELD

The present invention relates to a cap sterilizing and washing method and a device therefor, and more particularly to a cap sterilizing and washing method of a chute type that is advantageous for sterilizing and washing synthetic resin caps, while conveying the caps by a chute, and to a device therefor.

### BACKGROUND ART

When synthetic resin caps are fitted on bottle containers filled with contents such as refreshing beverages, a cap sterilizing and washing process is often implemented before the caps are fed to a capper. A method for sterilizing and washing the caps, while conveying them under gravity by a chute (see, for example, Patent Document 1), and a method for sterilizing and washing the caps, while conveying by a power drive, such that uses conveying with a turret rotationally driven about a horizontal axis or a drum-shaped star wheel (see, for example, Patent Documents 2 and 3) are known as cap washing and sterilizing methods of this kind. With these methods, hot water, or steam, or a sterilizing agent are jetted out onto the caps during conveying to sterilize the caps and then cooling and washing are conducted by jetting out germ-free water.

A cap conveying chute that is used to sterilize and wash during conveying by a chute is constituted so that a continuous cap conveying space is demarcated by a plurality of linear guides disposed at least above and below and on the left and right sides, so as to support the caps rotatably transversely, and the caps fall under gravity in a state in which they form a continuous row inside the chute. Sterilization and washing are performed by jetting out hot water or germ-free water (a case in which they are combined as hot water will be considered hereinbelow as a representative example) towards the outer circumferential surface and inner circumferential surface of the caps from nozzles disposed with an appropriate spacing above and below and/or on a side along the conveying path in the course of conveying the caps by the chute. In this case, the orientation of nozzles disposed along the chute is such that hot water is jetted out obliquely with respect to the conveying direction so as not to inhibit the conveying by the chute.

Patent Document 1: Japanese Patent Application Laid-Open No. 09-278087

Patent Document 2: Japanese Patent Application Laid-Open No. 11-193009

Patent Document 3: Japanese Patent Application Laid-Open No. 2003-128023

### DISCLOSURE OF THE INVENTION

### Problem to be Solved by the Invention

The above-described conventional method and device in which sterilization and washing are conducted during conveying under gravity by a chute are superior to the method and device in which sterilization and washing are conducted while conveying by power such as a turret in that no special power is required for conveying the caps, the equipment is simple, and the equipment cost is low. However, the following problems are yet to be resolved.

(1) The caps move in a state of row, while hot water is being jetted out thereon, and therefore receive an integral pressure and are gradually heated by the hot water. The feed of the caps by a feed-out turret in the vicinity of the outlet is restricted and the caps receive the maximum integral pressure in a state in which they are heated and softened when stopped. As a result, the skirt portion can be deformed into an elliptic shape.

(2) In the sterilizing and washing process, hot water or germ-free water is jetted out from the upstream side towards the downstream side onto the caps in a descending chute. Therefore, new germ-free water is gradually jetted out on the downstream side, as the hot water or germ-free water that has been jetted out upstream and contaminated moves together with the caps and remains sufficiently retained in and adhered to the caps. As a result, washing efficiency is decreased.

The present invention aims to resolve the above-described problems of the cap sterilizing and washing method by which a cap is sterilized and washed, while being conveyed by the conventional chute, and a device therefor, and it is an object of the present invention to provide a cap sterilizing and washing method and device that make it possible to prevent the cap from deformation by relaxing the integral pressure acting upon the synthetic resin cap inside the chute, reduce the effect produced by hot water or germ-free water jetted out on the upstream side on hot water or germ-free water on the downstream side, conduct sterilization and washing with good efficiency, and save space.

### Means for Solving the Problem

The cap sterilizing and washing method in accordance with the present invention that resolves the above-described problems is a method of sterilizing and washing a cap by jetting out a sterilizing and washing medium by a jet nozzle onto the cap conveyed by a chute, wherein the chute has an ascending chute portion, the sterilizing and washing medium is jetted out onto the cap that has been fed to the ascending chute portion, and the cap is sterilized and washed, imparted with a thrust for ascending the ascending chute and conveyed by the jet pressure of the sterilizing and washing medium.

Hot water, steam, a sterilizing agent, and germ-free water can be used as the sterilizing and washing medium, and hot water is especially preferred. The hot water is preferably jetted out at least towards an inner surface of an opening portion of the cap inside the chute at a jet angle of 0 to 120° and a jet pressure of 0.1 to 0.25 MPa, and in the usual sterilization and washing, 110° and a jet pressure of 0.2 MPa ensure good sterilization and washing efficiency and are preferred. Further, since the surface area of the cap that is bombarded by the washing medium jetted out from the nozzle decreases as the jet angle decreases, the washing medium is concentrated accordingly and the sterilization and washing performance is improved, but the consumption of the sterilizing medium increases. Therefore, more effective sterilization and washing can be performed by using jet nozzles that differ in a jet angle correspondingly to the sterilizing and washing zone of the cap or by arranging nozzles that differ in a jet angle (for example, fan-shaped nozzles with a jet angle of 110° and linear nozzles with a jet angle of)0° so that the nozzles are mixed at an appropriate ratio.

The cap sterilizing and washing device in accordance with the present invention that resolves the above-described problems is a cap sterilizing and washing device of a chute type in which a cap that is being conveyed by a chute is sterilized and washed by jetting out a sterilizing and washing medium by a jet nozzle onto the cap, wherein the chute has an ascending chute portion in which a conveying path forms a rising slope with an inclination angle α of more than 0° and equal to or less than 15° in a conveying direction with respect to a horizontal plane, and a sterilizing and washing medium pipe having a plurality of the jet nozzles that jet out the sterilizing and washing medium is disposed along the ascending chute portion. The chute has the ascending chute portions in a plurality of stages, and is disposed in a zigzag manner in a height direction by connecting the ascending chute portions by vertical descending chute portions, and the distance between an upstream end of the ascending chute of the upper stage and a downstream end of the ascending chute of the lower stage that immediately follows the upper stage is less than the distance between a downstream end of the ascending chute of the upper stage and an upstream end of the downstream ascending chute.

It is desirable that the jet nozzles be disposed at an inclination angle of 20° to 120° with respect to an axial line of the ascending chute portion in the conveying direction, and at least ⅔ of the plurality of jet nozzles be disposed at an inclination angle of 30° to 60°, preferably at 45°. Further, it is preferred that the sterilizing and washing medium pipe be composed of a combination of a sterilizing and washing medium pipe provided with skirt wall upper-side jet nozzles that jet out hot water from above towards a skirt wall of the cap, a sterilizing and washing medium pipe provided with skirt wall lower-side jet nozzles that jet out hot water from below towards the skirt wall of the cap, a sterilizing and washing medium pipe provided with inner jet nozzles that jet out hot water inward from an opening portion of the cap, and a sterilizing and washing medium pipe provided with a top wall jet nozzle that jets out hot water towards a top wall of the cap.

### EFFECTS OF THE INVENTION

In accordance with the inventions as described in claim 1 and claim 3, because the chute is formed as a rising slope, even though a thrust created by the jet pressure of the sterilizing and washing medium is received inside the chute, the transition to the turret disposed at the downstream end is restricted and a push-forward state is assumed, and integral pressure acting upon the cap is reduced from the thrust by a force component towards the upstream side in the conveying direction that is generated by gravity. Therefore, the integral pressure received by the cap is less than that in the conventional chute composed only of a descending slope and the deformation of the cap caused by hot water sterilization can be effective prevented.

Further, because the sterilizing and washing medium is jetted out onto the cap in the ascending chute portion, the sterilizing and washing medium that has been jetted out flows from the cap to the upstream side (that is in the direction opposite to the conveying direction) that is a descending slope. Therefore, the amount of the sterilizing and washing medium that has adhered to the cap surface and remains thereon is greatly reduced by comparison with the case of the descending chute in which the medium flows in the same direction as the conveying direction of the cap, the amount of the new washing medium that is jetted out from the nozzle on the downstream side and directly acts upon the cap surface increases accordingly and the sterilizing and washing can be efficiently performed. In addition, the sterilizing and washing medium on the downstream side where the sterilizing and washing have further advanced contributes to the sterilizing and washing of the caps on the upstream side and therefore the consumption of the sterilizing and washing medium can be reduced.

In accordance with the invention as in claim 2, the thrust can be effectively provided to the cap and the cap can be successfully conveyed by the jet pressure only even on the rising slope. Further, the inner surface of the cap that requires the largest degree of sterilizing and washing can be effective sterilized and washed.

In accordance with the invention as in claim 4, when the chute is disposed in a plurality of stages in a zigzag manner, the relationship between the chutes is such that a chute of the lower stage is disposed in a direction of decreasing the distance between the chutes towards the downstream side with respect to a chute of the upper stage. Therefore, a vertically compact structure can be realized, sufficient sterilizing and washing time can be ensured, and space can be saved.

In accordance with the invention as in claim 5, for the jet pressure of the sterilizing and washing medium from the nozzles to provide a thrust to the cap and convey the cap along the ascending chute, it is necessary to incline the jet nozzles with respect to the conveying direction so that the force component of the jet pressure contributes to the thrust. However, it is not necessary to incline all the nozzles in the conveying direction. Thus, the conveying speed and conveying distance can be controlled and better sterilizing and washing can be performed by admixing the nozzles with an inclination angle of equal to or greater than 90° C., which create a resistance in the conveying direction, at a ratio that does not inhibit good conveying. Further, in accordance with the invention as in claim 6, the washing medium jet pipes are disposed at four sides along the conveying path of the caps, thereby enabling more complete sterilizing and washing of the caps and more adequate use in a germ-free filling line.

### BRIEF DESCRIPTION OF THE DRAWINGS

FIG. 1 is a principal front view of the cap sterilizing and washing device according to the embodiment of the present invention;

FIG. 2 is a principal plan view thereof;

FIG. 3 is an enlarged view taken along the A-A arrow in FIG. 1;

FIG. 4 is a schematic diagram illustrating the arrangement mode of nozzles in examples and comparative examples;

FIG. 5 is a schematic diagram illustrating a jet pipe and the arrangement of jet nozzles in Example 1. In this figure, the position of the hot-water pipe C where the inner sterilizing nozzle is disposed is lowered below the actual arrangement position so as to prevent the cap from being hidden. The same is true for the below-described FIGS. 6 and 7;

FIG. 6 is a schematic diagram illustrating a jet pipe and the arrangement of jet nozzles in Example 2; and

FIG. 7 is a schematic diagram illustrating a jet pipe and the arrangement of jet nozzles in comparative example.

### EXPLANATION OF REFERENCE NUMERALS

1 cap sterilizing and washing device
5 chute
6 inlet vertical chute portion
7 first ascending chute portion
8 inner vertical chute portion
9 second ascending chute portion
10 outlet vertical chute portion
11, 12 feed-out turret
13 linear guide
14 chamber
15 base frame
16 bracket
17 support frame
18 hot water receiving pan
20 skirt wall upper-side jet nozzle
21, 23, 25, 27 hot water pipe
22 skirt wall lower-side jet nozzle
24 inner jet nozzle
24-1 fan-shaped nozzle
24-2 straight forward nozzle
26 top wall jet nozzle
30 hot water
35 synthetic resin cap
36 tamper evident band
37 slit

### BEST MODE FOR CARRYING OUT THE INVENTION

An embodiment of the present invention will be described below in greater detail with reference to the appended drawings.

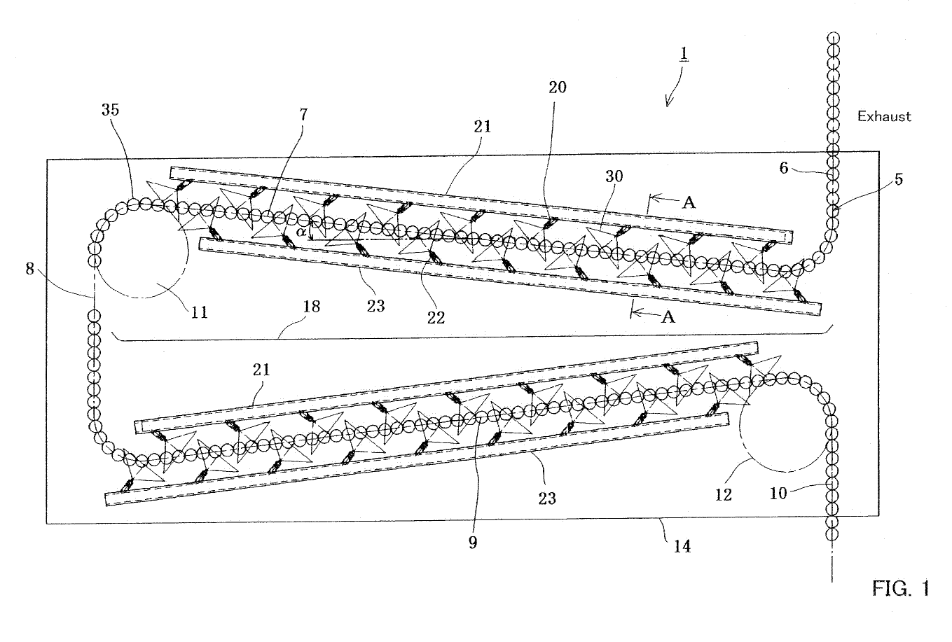

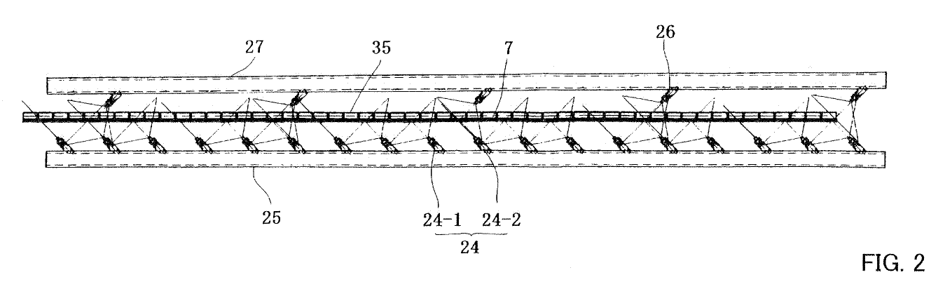

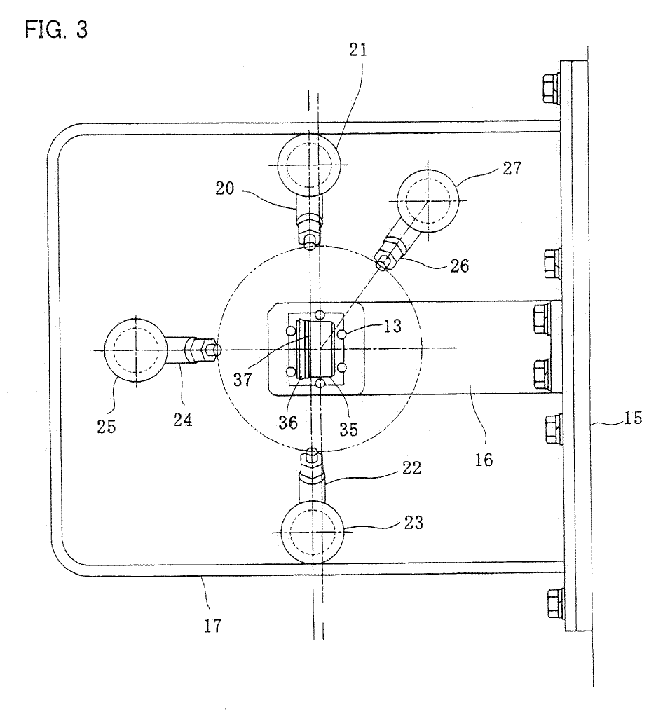

FIG. 1 to FIG. 3 show a cap sterilizing and washing device of a chute conveying system according to an embodiment of the present invention. FIG. 1 is a front schematic view of the device, FIG. 2 is an upper view thereof, and FIG. 3 is a side enlarged view thereof.

A major specific feature of the cap sterilizing and washing device of a chute conveying system in accordance with the present invention that differentiates this device from the conventional cap sterilizing and washing device of a chute conveying system is that a chute ascends rather than descends with respect to the conveying direction and a conveying force of a cap is obtained from a force component of hot water jetted out from a nozzle or a jet pressure of washing water. Cap sterilization can be performed by sterilizing and washing with hot water, steam, or a sterilizing agent, and a jet of washing water is necessary to cool after the sterilization in the case of sterilizing with hot water and steam and to remove a sterilizing agent in the case of sterilizing with the sterilizing agent. In the below-described embodiment, only a process in which sterilizing and washing are performed by jetting out hot water will be explained, but the present invention is not limited to such a process.

In the present embodiment, as shown in FIG. 1, a chute 5 is disposed as a whole in a zigzag manner in the vertical direction, and a cap 35 that has been fed from above is conveyed downward, sterilized and washed with hot water in the conveying process, and discharged downward. The chute is constituted by an inlet vertical chute portion 6, a first ascending chute portion 7, an inner vertical chute portion 8, a second ascending chute portion 9, and an outlet vertical chute portion 10. A plurality of hot water pipes serving as sterilizing and washing medium pipes having disposed therein a group of jet nozzles that jet out hot water is disposed along the first ascending chute portion 7 and second ascending chute portion 9. The first ascending chute portion 7 and second ascending chute portion 9 both ascend with an inclination angle α with respect to a horizontal surface toward the downstream side. Therefore, as shown in the figure, the first ascending chute portion 7 and second ascending chute portion 9 are inclined in the mutually opposite directions, and when they are provided in a multistage configuration, both sides are alternately inclined inside in the height direction and a distance between the two sloping chutes is reduced thereby making it possible to obtain a compact configuration in the vertical direction. In the present embodiment, only two ascending chute portions are provided, but if necessary, a multistage configuration including three or more stages may be used.

Feed-out turrets 11, 12 for changing the conveying direction of the cap that has been conveyed along the rising slope trajectory by the chute to a vertical direction, feeding the cap to the inner vertical chute portion 8 and outlet vertical chute portion 10, and adjusting the feed-out speed of the cap are disposed at the downstream end (top portion) of the first ascending chute portion 7 and second ascending chute portion 9, respectively. The feed-out turrets 11, 12 have pockets for accommodating the sidewise tumbled caps at the outer circumference and are rotationally driven by a motor that is not shown in the figure. The caps that have reached the top of the ascending chute are accommodated in respective pockets and fed out. Therefore, by controlling the rotation speed of the feed-out turrets, it is possible to adjust freely the feed-out speed of caps that are fed in a row in a continuous manner.

The entire chute 5 is disposed inside a chamber 14 and the hot water or germ-free water jetted out inside the chamber is prevented from scattering to the outside of the device. As shown in FIG. 3, in the first ascending chute portion 7 and second ascending chute portion 9, brackets 16 that support a linear guide 13 composed of a circular line constituting the chute are disposed in a protruding condition in the horizontal direction with a predetermined spacing from a base frame 15 on which the chute portions are disposed in a vertical state at a respective predetermined inclination angle to the longitudinal direction. In the present embodiment, as shown in FIG. 3, the linear guide 13 is the linear guide 13 composed of a total of six linear guides: a pair of upper and lower linear guides that guide the central portion of the skirt wall of the cap from above and below, two linear guides that guide the opening portion, and two linear guides that guide the outer surface portion of the top wall, and these linear guides demarcate the conveying path through which the cap passes. A support frame 17 that supports the below-described hot water pipe is provided in a protruding condition from the base frame 15 so as to surround the conveying path.

In the present embodiment, as shown in FIG. 3, the hot water pipe is composed of a total of four pipes: a hot water pipe 21 provided with a skirt wall upper-side jet nozzle 20 that jets out hot water from above towards the skirt wall of the synthetic resin cap 35, a hot water pipe 23 having disposed therein a skirt wall lower-side jet nozzle 22 that jets out hot water from below towards the skirt wall of the cap, a hot water pipe 25 provided with an inner jet nozzle 24 that jets out hot water inward from the opening portion of the cap, and a hot water pipe 27 provided with a top wall jet nozzle 26 that jets out hot water towards the top wall of the cap. The hot water pipes are mounted on the support frame 17 by appropriate brackets. However, the above-described arrangement of hot-water pipes is not limiting. For example, the hot water pipe 27 that jets out hot water on the top wall surface of the cap can be omitted.

The inclination angle α of the rising slope of the first ascending chute portion 7 and second ascending chute portion 9 may be any angle, provided that the cap can be smoothly conveyed against the force of gravity by the below-described conveying force of the cap obtained by jetting out the hot water, and the preferred angle is within a range of more than 0° and equal to or less than 15°. Where the inclination angle is above 15°, the slope is steep, a high-pressure hot water jet is required, and the cap is difficult to convey smoothly. Further, a large amount of hot water is necessary and the process is cost inefficient. As for the lower limit angle, if the inclination is too small, the conveying speed resistance is small and the benefits of the rising slope are reduced. Therefore, it is preferred that the angle be at least larger than 0°, preferably equal to or greater than 3°.

The hot water pipes are disposed along the chute at the same inclination as the ascending chute portions, but it is preferred that each pipe be disposed in the following positions.

Thus, it is preferred that the hot water pipes 21, 23 be provided in positions that are offset downward from a center of the skirt wall in the height direction, as shown in FIG. 3, so that the skirt wall upper-side jet nozzle 20 and skirt wall lower-side jet nozzle 22 jet out hot water towards a slit (weakening line) 37 provided between the lower end of the skirt portion of the synthetic resin cap and a tamper evident band 36. As a result, it is possible to sterilize and wash reliably the slit portion to which contaminants and germs easily adhere. Further, it is preferred that the hot water pipe 25 provided with the inner jet nozzle 24 be provided in the central portion at the side surface of the conveying path, as shown in FIG. 3, so that hot water could be jetted from the center of the opening portion of the cap that passes along the conveying path onto the inner surface of the top wall. Further, from the standpoint of effective sterilization, it is preferred that the hot water pipe 27 provided with the top wall jet nozzle 26 be provided in a position shifted from the central portion of the top wall, so that hot water could be fed obliquely with respect to the conveying direction from the outer circumferential portion of the top wall of the cap that passes along the conveying path as shown in FIG. 3.

The types and inclination angles of the jet nozzles disposed in each hot water pipe are important for efficiently sterilizing the cap and imparting a conveying force to the cap. In the present embodiment, the skirt wall upper-side jet nozzles 20 and skirt wall lower-side jet nozzles 22 are provided in the hot water pipes 21, 23, respectively, with the same pitch and same inclination angle with respect to the cap conveying direction from the upper side and lower side, as shown in FIG. 1. The hot water pipe 21 on the upper side and the hot water pipe 23 on the lower side are disposed with a displacement such that the skirt wall upper-side jet nozzles 20 and skirt wall lower-side jet nozzles 22 are displaced by half a pitch to ensure alternate arrangement. As shown schematically in FIG. 1, the skirt wall upper-side jet nozzles 20 and skirt wall lower-side jet nozzles 22 use fan-shaped nozzles that jet out a fan-shaped jet of hot water 30, such that the skirt wall surface, in particular the slit portion, could be washed. As for the inclination angle of the skirt wall upper-side jet nozzles 20 and skirt wall lower-side jet nozzles 22 with respect to the central line of the conveying path, the angle is also affected by the inclination angle of the chute, but the nozzles can be disposed with an inclination angle of 20° to 120° with respect to the axial line of the ascending chute portion in the conveying direction.

In order to obtain a thrust from the hot water jet, the inclination angle of nozzles with respect to the conveying direction in theory can be within a range 0° to 90°, but where this angle is equal to or less than 20°, the arrangement of nozzles decreases the amount of jetted hot water that falls on the cap, the cap is difficult to convey effectively, the hot water is wasted, and the sterilization efficiency decreases. Where the angle exceeds 90°, the pressure of jetted hot water creates a resistance with respect to the conveying direction and the thrust decreases. However, by using this effect, it is also possible to create a resistance to a cap moving at a high speed, reduce this speed, and provide a gap between the caps by using a mixed arrangement with nozzles having an inclination angle of equal to or greater than 90°. Accordingly, the maximum range of the nozzle inclination angle was taken as 120°. Where the inclination angle of 120° is exceeded, the conveying force and washing efficiency are degraded in the same manner as in the case in which the inclination angle with respect to the conveying direction is equal to or less than 20°. However, when the nozzles are disposed with an inclination angle of equal to or greater than 90°, the resistance increases and the cap cannot be effectively conveyed unless that number of such nozzles is equal to or less than about ⅓ of the number of nozzles having the inclination angle in the propulsion direction. Therefore, it is desirable that the arrangement be within this range or below it.

The inner jet nozzles 24 are disposed with a pitch less than that of the skirt wall upper-side jet nozzles 20 and skirt wall lower-side jet nozzles 22 and a larger amount of hot water is jetted out from the inner jet nozzles on the inner surface of the cap in order to sterilize and wash the inside of the cap that requires the highest level of sterilization. Nozzles of two types, namely, fan-shaped nozzles 24-1 that jet out a fan-shaped jet of hot water and straight-forward nozzles 24-2 that jet out a linear straight-forward jet of hot water are used as the inner jet nozzles 24, and one straight-forward nozzle is disposed for every two fan-shaped nozzles 24-1. By arranging the straight-forward nozzles in such a manner, it is possible to increase the washing ability of deep portions of the cap. The top wall jet nozzles jet out hot water on the outer surface of the top wall and sterilize and wash the outer surface of the top wall. Because the outer surface of the top wall is generally flat and the easiest to sterilize, it is possible to dispose the smaller number of the top wall jet nozzles and with a larger pitch as compared with the above-described nozzles that sterilize and wash other locations. In the present embodiment, as shown in FIG. 2, the fan-shaped nozzles are disposed with a pitch that is four times that of the inner jet nozzles, but such a configuration is not limiting.

Further, in the present embodiment, a hot water receiving pan 18 is provided between the first ascending chute portion 7 and the second ascending chute portion, thereby making it possible to receive the hot water jetted out on the conveyed cap and recover the hot water so that the contaminated hot water does not reach the ascending chute of the lower stage. As a result, although the ascending chutes are provided in a plurality of stages, only new hot water jetted out from the jet nozzles acts upon the cap that is conveyed along each ascending chute and the sterilizing and washing effect is increased.

### EXAMPLES

### Example 1

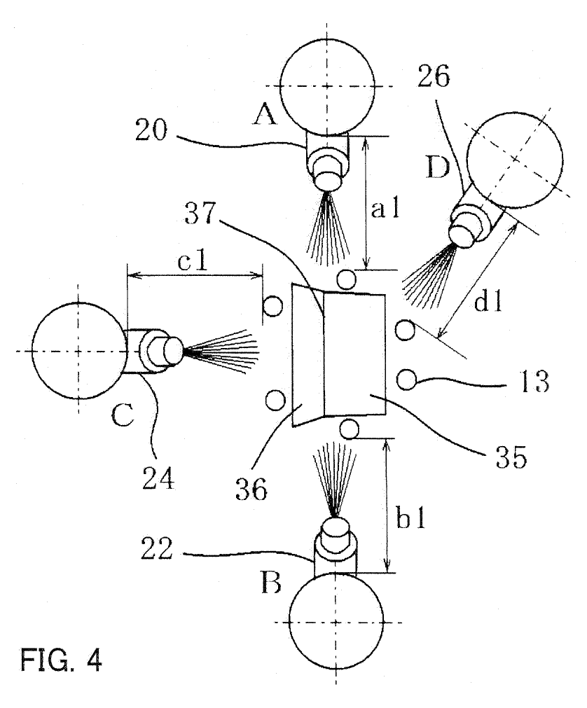

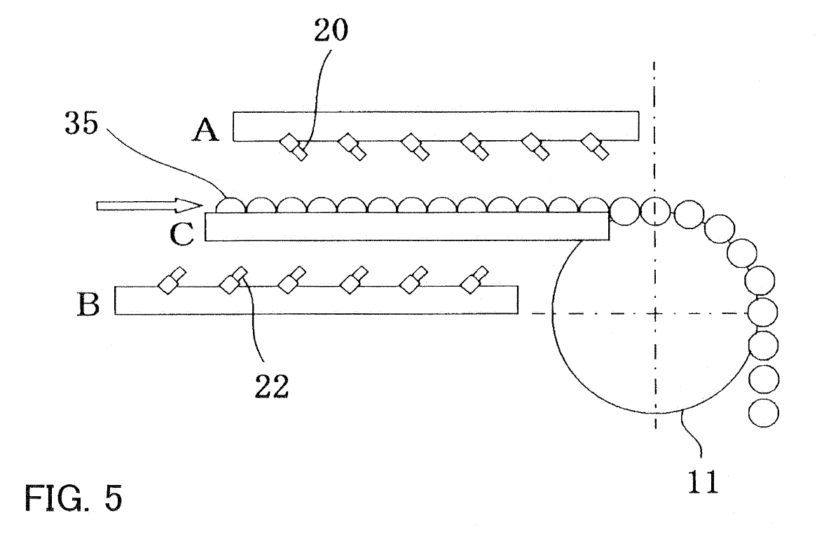

A chute was configured by setting an inclination angle of an ascending chute portion to 7°, and hot water pipes A to D were disposed around the chute in an arrangement such as shown in FIG. 4. These hot water pipes A, B, C, and D had a length (nozzle segment length 1400 mm) such that a maximum of 14 nozzles could be disposed along the entire length on the basis of a 100-mm pitch, and the jet nozzles were disposed in respective locations from the upstream side in a state shown in Table 1. In the table, F stands for a fan-shaped nozzle (jet angle 110°), S stands for a straight-forward nozzle (jet angle 0°), and P stands for a plug (that is, closed state without a nozzle). All the jet nozzles were disposed at an angle of 45° with respect to the conveying direction as shown in FIG. 5. In FIG. 4, a1, b1, c1, and d1 indicate a linear distance from the outer circumference of each hot water pipe to the linear guide in the proximity of the chute. In the present embodiment, a1=b1=c1=85 mm and d1=80 mm.

TABLE 1
From upstream side (P: plug, F: fan-shaped
nozzle, S: straight-forward nozzle)

| | 1 | 2 | 3 | 4 | 5 | 6 | 7 | 8 | 9 | 10 | 11 | 12 | 13 | 14 |
|---|---|---|---|---|---|---|---|---|---|---|---|---|---|---|
| A | F | P | F | P | F | P | F | P | F | P | F | P | F | P |
| B | P | F | P | F | P | F | P | F | P | F | P | F | P | F |
| C | F | S | F | F | S | F | F | S | F | F | S | F | F | S |
| D | F | P | P | F | P | P | F | P | P | F | P | P | F | P |

Total number of nozzles: 33

In the above-described device, in order to confirm a washing effect, red water (1% aqueous solution) was blown onto a synthetic resin cap having a tamper evident band 36 via a slit 37 at the lower end of a skirt wall, and a cap in which a slit portion was contaminated and became red was taken as a sample and supplied to the ascending chute portion. The operations were performed under the test conditions shown in Table 2 and the sterilizing and washing effect and conveying state were visually confirmed. The results obtained demonstrated that the cap had effectively passed through the ascending chute portion, and although contamination with red water remained in the form of slight adhesion to the slit portion of the cap, the contamination was completely removed from the slit portion in the second pass. Therefore, it was confirmed that in the present embodiment good sterilizing and washing could be performed by doubling the nozzle segment length.

TABLE 2

| Jet | Total flow | Pump | Turret rotation | Treatment | Passage |
|---|---|---|---|---|---|
| pressure, | rate, | frequency, | speed, | speed, | time, |
| MPa | L/min | Hz | rpm | cpm | sec |
| 0.200 | 32.01 | 34 | 30 | 720 | 3.5 |

### Example 2

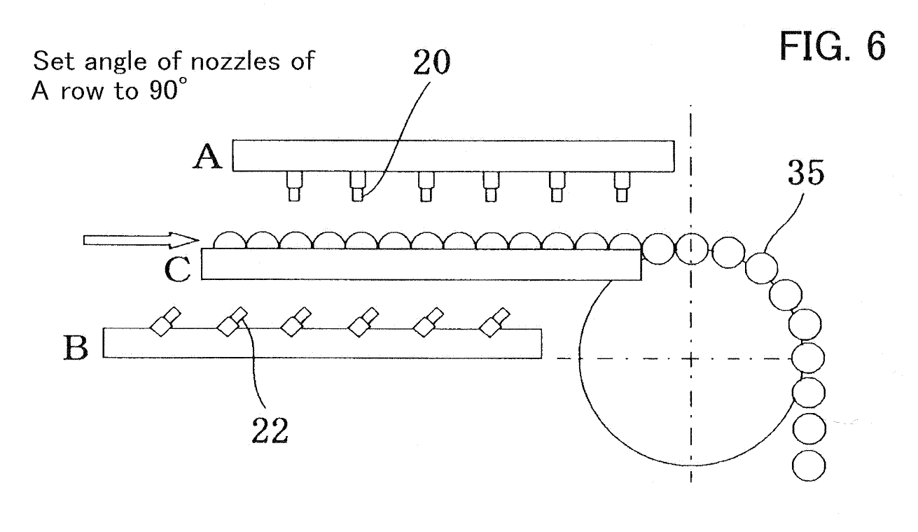

In Example 2, all the jet nozzles of the hot water pipe A were disposed so as to be at a right angle to the conveying direction, as shown in FIG. 6. In this case, the total number of jet nozzles having an inclination angle of 90° was seven, which was about 21% of the total number (33) of the jet nozzles. In this case, hot water jetted out from the jet nozzles perpendicular to the conveying direction and disposed in the hot water pipe A created jet pressure acting upon the cap that became conveying resistance and an opening appeared between the conveyed caps. As a result, shadow portions onto which the hot water did not fall were eliminated, the hot water fell effectively on the entire periphery of the cap and the washing effect was increased over that in Example 1. Therefore, the orientation of nozzles is not limited to that in which all the nozzles are disposed obliquely with respect to the conveying direction of the cap. Thus, it was confirmed that by disposing the nozzles with orientation that creates resistance to conveying of caps in some of the pipes and creating a resistance to a degree that does not affect the cap conveying and processing speed, as in the present embodiment, it is possible to increase the washing efficiency and save hot water.

### Comparative Example 1

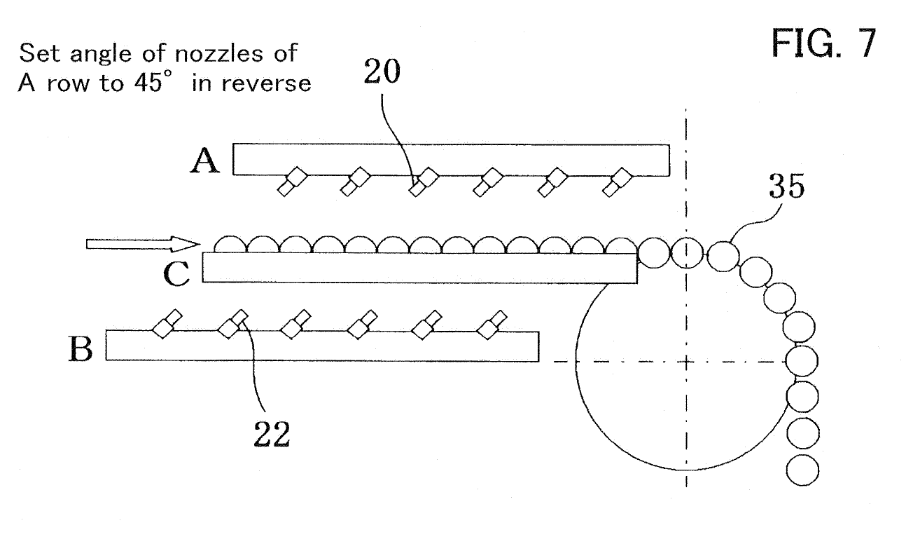

In Comparative Example 1, the test was conducted in the same manner as in Example 1 by setting the angle of nozzles in the hot water pipe A to 45° in reverse to the conveying direction, as shown in FIG. 7. In Comparative Example 1, setting the nozzles of A row to 45° in reverse was expected to impart a rotation force to the cap and increase the washing efficiency of the slit, but in this case no thrust to the cap was obtained and the cap stopped and did not move.

### INDUSTRIAL APPLICABILITY

The cap sterilizing and washing method and device in accordance with the present invention make it possible to relax an integral pressure acting upon a synthetic resin cap in a chute and prevent the cap from deformation, reduce the effect produced by hot water or germ-free water jetted out on the upstream side on hot water or germ-free water on the downstream side, and perform sterilizing and washing with good efficiency. Therefore, these method and device are particularly advantageous for sterilizing and washing synthetic resin caps having a tamper evident band. However, this application is not limiting and the method and apparatus can be applied to sterilizing and washing a variety of caps and can be also advantageously used in aseptic filling and sealing lines that require a level of sterilizing and washing higher than the usual filling and sealing lines.

1. A method of sterilizing and washing a cap by jetting out a sterilizing and washing medium by a jet nozzle onto the cap conveyed by a chute, wherein the chute has an ascending chute portion, the sterilizing and washing medium is jetted out onto the cap that has been fed to the ascending chute portion, and the cap is sterilized and washed, imparted with a thrust for ascending the ascending chute and conveyed by the jet pressure of the sterilizing and washing medium.

2. The method of sterilizing and washing a cap according to claim 1, wherein the sterilizing and washing medium is hot water, and the hot water is jetted out by the jet nozzle at least towards an inner surface of an opening portion of the cap inside the ascending chute portion at a jet angle of 0 to 120° and a jet pressure of 0.1 to 0.25 MPa.

3. A cap sterilizing and washing device of a chute type in which a cap that is being conveyed by a chute is sterilized and washed by jetting out a sterilizing and washing medium by a jet nozzle onto the cap, wherein the chute has an ascending chute portion in which a conveying path forms a rising slope with an inclination angle α of more than 0° and equal to or less than 15° in a conveying direction with respect to a horizontal plane, and a sterilizing and washing medium pipe having a plurality of the jet nozzles that jet out the sterilizing and washing medium is disposed along the ascending chute portion.

4. The cap sterilizing and washing device according to claim 3, wherein the chute has the ascending chute portions in a plurality of stages, and is disposed in a zigzag manner in a height direction by connecting the ascending chute portions by vertical descending chute portions, and the distance between an upstream end of the ascending chute of the upper stage and a downstream end of the ascending chute of the lower stage that immediately follows the upper stage is less than the distance between a downstream end of the ascending chute of the upper stage and an upstream end of the downstream ascending chute.

5. The cap sterilizing and washing device according to claim 3, wherein the jet nozzles are disposed at an inclination angle of 20° to 120° with respect to an axial line of the ascending chute portion in the conveying direction, and at least ⅔ of the plurality of jet nozzles are disposed at an inclination angle of 30° to 60°.

6. The cap sterilizing and washing device according to claim 3, wherein the sterilizing and washing medium pipe comprises a combination of a sterilizing and washing medium pipe provided with skirt wall upper-side jet nozzles that jet out hot water from above towards a skirt wall of the cap, a sterilizing and washing medium pipe provided with skirt wall lower-side jet nozzles that jet out hot water from below towards the skirt wall of the cap, a sterilizing and washing medium pipe provided with inner jet nozzles that jet out hot water inward from an opening portion of the cap, and a sterilizing and washing medium pipe provided with a top wall jet nozzle that jets out hot water towards a top wall of the cap.

7. The cap sterilizing and washing device according to claim 4, wherein the jet nozzles are disposed at an inclination angle of 20° to 120° with respect to an axial line of the ascending chute portion in the conveying direction, and at least ⅔ of the plurality of jet nozzles are disposed at an inclination angle of 30° to 60°.

8. The cap sterilizing and washing device according to claim 4, wherein the sterilizing and washing medium pipe comprises a combination of a sterilizing and washing medium pipe provided with skirt wall upper-side jet nozzles that jet out hot water from above towards'a skirt wall of the cap, a sterilizing and washing medium pipe provided with skirt wall lower-side jet nozzles that jet out hot water from below towards the skirt wall of the cap, a sterilizing and washing medium pipe provided with inner jet nozzles that jet out hot water inward from an opening portion of the cap, and a sterilizing and washing medium pipe provided with a top wall jet nozzle that jets out hot water towards a top wall of the cap.
